(19) United States
(12) Patent Application Publication
(10) Pub. No.: US 2008/0269583 A1
(43) Pub. Date: Oct. 30, 2008

(54) Detection and Monitoring of Stress Events During Sleep

(75) Inventor: Daniel Reisfeld, Tel-Aviv (IL)

Correspondence Address: WOLF GREENFIELD & SACKS, P.C., 600 ATLANTIC AVENUE, BOSTON, MA, 02210-2206, US

(73) Assignee: WIDEMED LTD., Herzlia (IL)

(21) Appl. No.: 11/883,471

(22) PCT Filed: Feb. 7, 2006
(86) PCT No.: PCT/IL06/00148

§ 371 Date: Jun. 11, 2008

Related U.S. Application Data
(60) Provisional application No. 60/651,295, filed on Feb. 7, 2005.

(51) Int. Cl. A61B 5/087; A61B 5/11; A61B 5/02; A61B 5/145

(52) U.S. Cl. 600/364; 600/538; 600/534; 600/484

(57) ABSTRACT

A computer-implemented method for monitoring a patient (22) includes receiving physiological signals from the patient during sleep and processing at least one of the signals to detect a spontaneous stress event. One or more of the signals following the stress event are analyzed so as to evaluate a stress response of the patient.

### CROSS-REFERENCE TO RELATED APPLICATION

This application claims the benefit of U.S. Provisional Patent Application 60/651,296, filed Feb. 7, 2005, which is incorporated herein by reference.

### FIELD OF THE INVENTION

The present invention relates generally to physiological monitoring and diagnosis, and specifically to sleep recording and analysis.

### BACKGROUND OF THE INVENTION

Sleep apneas commonly occur in conjunction with a variety of cardiorespiratory disorders. The relationship between sleep apnea and heart failure, for example, is surveyed by Bradley et al. in two articles entitled “Sleep Apnea and Heart Failure,” including “Part I: Obstructive Sleep Apnea,” Circulation 107, pages 1671-1678 (2003), and “Part II: Central Sleep Apnea,” Circulation 107, pages 1822-1826 (2003), which are incorporated herein by reference. The authors define “apnea” as a cessation of airflow for more than 10 sec. This term is distinguished from “hypopnea,” which is a reduction in but not complete cessation of airflow to less than 50% of normal, usually in association with a reduction in oxyhemoglobin saturation (commonly referred to as “desaturation”).

Sleep apneas and hypopneas are generally believed to fall into two categories: obstructive, due to collapse of the pharynx; and central, due to withdrawal of central respiratory drive to the muscles of respiration. Central sleep apnea (CSA) is commonly associated with Cheyne-Stokes respiration, which is a form of periodic breathing in which central apneas and hypopneas alternate with periods of hyperventilation, with a waxing-waning pattern of tidal volume. CSA is believed to arise as the result of heart failure, but obstructive sleep apnea (OSA) may also occur in heart failure patients.

Both OSA and CSA increase the strain on the cardiovascular system and thus worsen the prognosis of the heart failure patient. In some cases, both types of apneas may occur in the same patient, even at the same time (superposition). Classifying respiratory events as central or obstructive is considered to be a critical point, since treatment may differ according to the type of events, as pointed out by Pepin et al. in “Cheyne-Stokes Respiration with Central Sleep Apnea in Chronic Heart Failure: Proposals for a Diagnostic and Therapeutic Strategy,” Sleep Medicine Reviews 10, pages 33-47 (2006), which is incorporated herein by reference. Both CSA and OSA can be manifested in periodic breathing patterns.

Various methods have been proposed in the patent literature for automated apnea detection and diagnosis based on patient monitoring during sleep. For example, U.S. Patent Application Publication US 2004/0230105 A1 describes a method for analyzing respiratory signals using a Fuzzy Logic Decision Algorithm (FLDA). The method may be used to associate respiratory disorders with obstructive apnea, hypopnea, central apnea, or other conditions. As another example, U.S. Patent Application Publication US 2002/0002327 A1 describes a method for detecting Cheyne-Stokes respiration in patients with congestive heart failure. The method involves performing spectral analysis of overnight oximetry recordings, from which a classification tree is generated. Another method, based on monitoring oxygen saturation and calculating the slope of desaturation events, is described in U.S. Pat. No. 6,760,608. Yet another method for classifying sleep apneas is described in U.S. Pat. No. 6,856,829. In this case, pulse waves from the body of a patient are detected, and the envelope of the pulse waves is created by connecting every peak of the pulse waves. The normalized amplitude and period of the envelope are used in determining whether the patient has OSA, CSA, or mixed sleep apnea syndrome. -The disclosures of the patents and patent applications cited above are incorporated herein by reference.

It has been suggested that sleep monitoring can be used for assessing cardiorespiratory risk. For example, U.S. Pat. No. 5,902,250, whose disclosure is incorporated herein by reference, describes a home-based, wearable, self-contained system that determines sleep-state and respiratory pattern, and assesses cardiorespiratory risk. A respiratory disorder may be diagnosed from the frequency of eyelid movements and/or from ECG signals. Cardiac disorders (such as cardiac arrhythmia or myocardial ischemia) that are known to be linked to certain respiratory disorders also may be inferred upon detection of such respiratory disorders. Stress effects on the heart that may occur during sleep are further described by Verrier et al., in “Sleep, Dreams, and Sudden Death; The Case for Sleep as an Autonomic Stress Test for the Heart,” Cardiovascular Research 31, pages 181-211 (1996), which is incorporated herein by reference. This article introduces the concept that the profound surges in autonomic activity that occur during sleep may constitute a diagnostic stress test capable of disclosing undocumented cardiac electrical instability.

### SUMMARY OF THE INVENTION

Embodiments of the present invention provide improved methods and systems for identifying spontaneous stress events during sleep and for analyzing the response of the patient's body to such stress events. The identification and response to the stress event may be used in diagnosing the patient's condition, as well as assessing prognosis and controlling treatment. Monitoring spontaneous stress events in this manner can be conducted automatically over extended periods while the patient is at home or in the hospital, as opposed to conventional laboratory stress testing, which requires dedicated equipment and a trained technician.

In an exemplary embodiment, a respiration-related signal may be processed to detect periodic breathing patterns during sleep. A shape characteristic of the periodic breathing pattern, such as the symmetry of the pattern, is used in classifying the etiology of the episode, by determining the episode to be predominantly obstructive or central in origin, for example. This classification is based on the inventors' discovery that Cheyne-Stokes respiration of purely central origin is characterized by a relatively symmetrical pattern of rise and fall in respiration parameters, by comparison with periodic breathing of obstructive origin.

There is therefore provided, in accordance with an embodiment of the present invention, a computer-implemented method for monitoring a patient, including:

receiving a signal associated with respiration of the patient during sleep;

processing the signal to detect a pattern of periodic breathing;

extracting from the signal a shape characteristic of the pattern; and

classifying an etiology of the periodic breathing responsively to the shape characteristic.

In a disclosed embodiment, classifying the etiology includes determining the periodic breathing to be predominantly central or obstructive in origin, wherein extracting the shape characteristic includes computing a parameter that is characteristic of a symmetry of the pattern. Typically, the periodic breathing is classified as predominantly central in origin if the pattern is symmetrical.

In disclosed embodiments, receiving the signal includes receiving an indication of least one of a flow through an airway of the patient, a movement of an abdomen or thorax of the patient, a heart rate of the patient, a respiration rate of the patient, a blood flow of the patient, a blood oxygen saturation level of the patient, and a pulse transit time (PTT) of the patient. In some embodiments, receiving the signal includes receiving a photoplethysmographic signal from an organ of the patient.

In some embodiments, the pattern includes a hyperventilation interval and a hypoventilation interval, and extracting the shape characteristic includes fitting first and second waveforms to the hyperventilation and hypoventilation intervals, respectively. Typically, the first and second wavefonns include analytical functions, which may include polynomial waveforms or tilted parabolic waveforms. In a disclosed embodiment, the method includes extracting features from the signal responsively to the first and second waveforms, and selecting a treatment to administer to the patient based on the extracted features.

There is also provided, in accordance with an embodiment of the present invention, a computer-implemented method for monitoring a patient, including:

receiving physiological signals from the patient during sleep;

processing at least one of the signals to detect a spontaneous stress event; and

analyzing one or more of the signals following the stress event so as to evaluate a stress response of the patient.

In some embodiments, the spontaneous stress event includes at least one of an apnea and a hypopnea.

In one embodiment, analyzing the one or more of the signals includes detecting a change in the signals relative to a baseline, and alerting at least one of the patient and a medical caregiver of the change.

In another embodiment, receiving the physiological signals includes collecting the signals from the patient at a first location where the patient is sleeping, and transmitting the signals over a communication network, and analyzing the one or more of the signals includes receiving the one or more of the signals over the communication network at a second location, remote from the first location, and analyzing the received signals at the second location.

There is additionally provided, in accordance with an embodiment of the present invention, apparatus for monitoring a patient, including:

a sensor, which is coupled to produce a signal associated with respiration of the patient during sleep; and

a diagnostic processor, which is arranged to process the signal to detect a pattern of periodic breathing, to extract from the signal a shape characteristic of the pattern, and to classify an etiology of the periodic breathing responsively to the shape characteristic.

There is further provided, in accordance with an embodiment of the present invention, apparatus for monitoring a patient, including:

a sensor, which is coupled to receive physiological signals from the patient during sleep; and

a diagnostic processor, which is arranged to process at least one of the signals to detect a spontaneous stress event, and to analyze one or more of the signals following the stress event so as to evaluate a stress response of the patient.

There is moreover provided, in accordance with an embodiment of the present invention, apparatus for monitoring a patient, including:

a belt, which is adapted to be fastened around a body of the patient; and

a sensor unit, which is coupled to the belt so as to sense a respiratory motion of the patient, and which includes a photoplethysmographic sensor, which is held against the body by the belt and is arranged to measure at least one of a blood flow and a blood oxygen saturation level of the patient.

Typically, the belt is configured to be fastened around a thorax of the patient, and the photoplethysmographic sensor includes a reflective sensor.

In some embodiments, the apparatus is used together with an electrocardiogram (ECG) signal that is received from a Holter monitor coupled to the patient.

The present invention will be more fully understood from the following detailed description of the embodiments thereof, taken together with the drawings in which:

### BRIEF DESCRIPTION OF THE DRAWINGS

FIG. 1A is a schematic, pictorial illustration of a system for patient monitoring during sleep, in accordance with an embodiment of the present invention;

FIG. 1B is a schematic, pictorial illustration showing a sensing device used in a system for patient monitoring during sleep, in accordance with another embodiment of the present invention;

FIGS. 2A and 2B are plots of physiological signals received from patients during sleep, in accordance with an embodiment of the present invention;

FIG. 3 is a flow chart that schematically illustrates a method for detection and analysis of stress events, in accordance with an embodiment of the present invention;

FIG. 4 is a plot showing model parameters used in analyzing a respiration-related signal, in accordance with an embodiment of the present invention; and

FIGS. 5A and 5B are plots showing curves fitted to respiration-related signals using the model of FIG. 4, in accordance with an embodiment of the present invention.

### DETAILED DESCRIPTION OF EMBODIMENTS

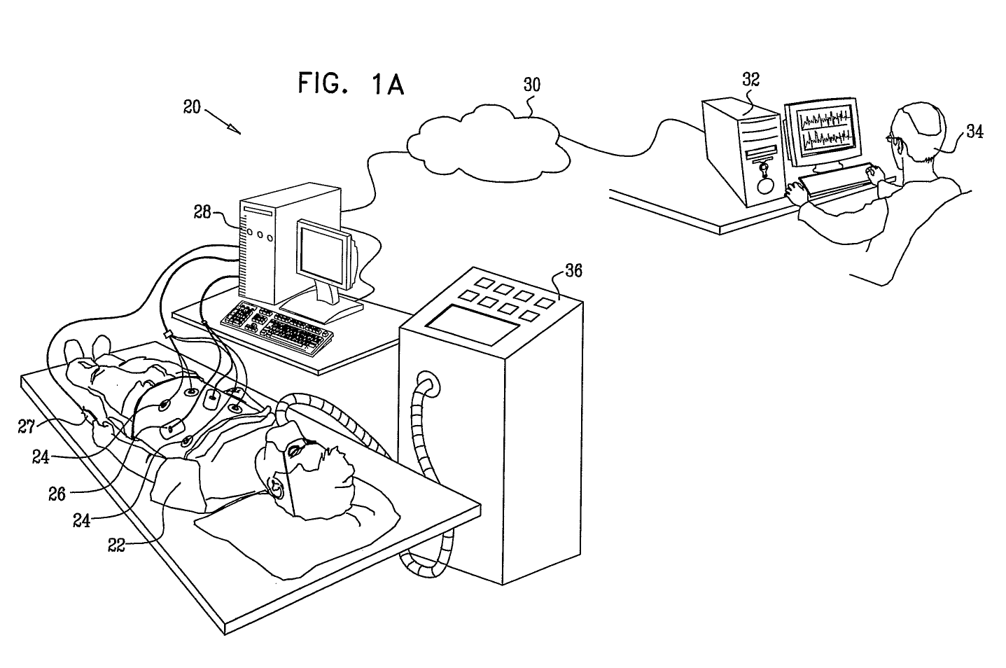

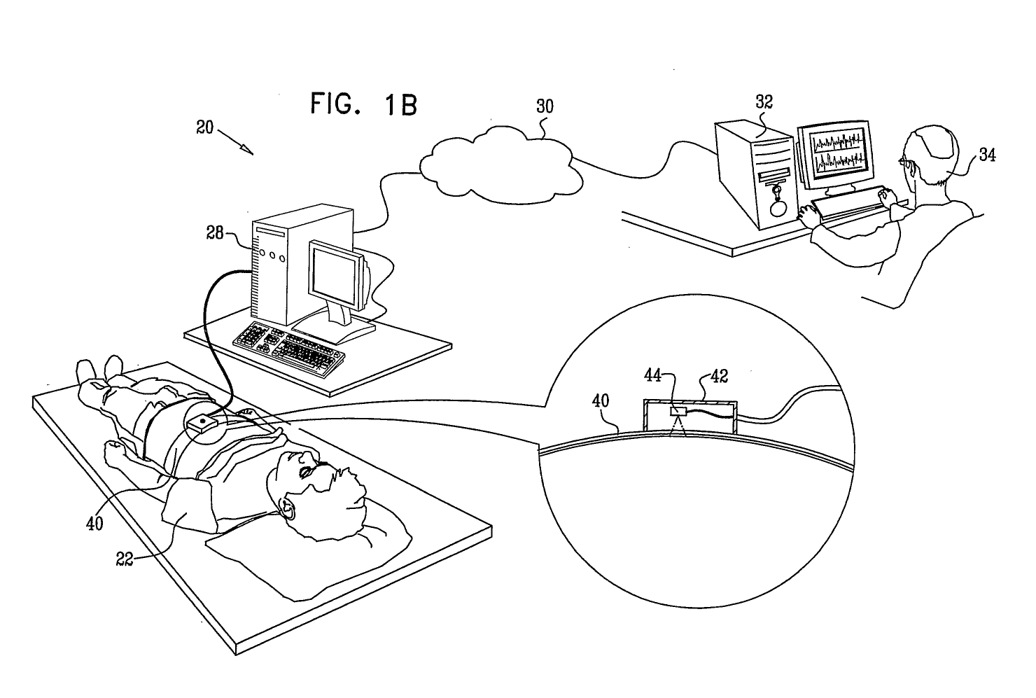

FIG. 1 is a schematic, pictorial illustration of a system 20 for sleep monitoring and diagnosis, in accordance with an embodiment of the present invention. In this embodiment, system 20 is used to monitor a patient 22 in a home, clinic or hospital ward environment, although the principles of the present invention may similarly be applied in dedicated sleep laboratories. System 20 receives and analyzes physiological signals generated by the patient's body, which may include an ECG signal measured by skin electrodes 24, which serve as ECG sensors, and a respiration signal measured by a respiration sensor 26. Additionally or alternatively, the system comprises a photoplethysmograph device 27, which serves as an oxygen saturation sensor. Device 27 provides a photoplethysmograph signal indicative of blood flow and a signal indicative of the level of oxygen saturation in the patient's blood. Since the photoplethysmograph signal is modulated by both the heart rate and respiratory rate, it may also be used to provide a heart rate and respiratory rate signals. The sensor signals are collected, amplified and digitized by a console 28. Although no EEG or EOG electrodes are shown in FIG. 1, the techniques of monitoring and analysis that are described herein may alternatively be combined with EEG, EOG, leg motion sensors, and other sleep monitoring modalities that are known in the art.

Respiration sensor 26 typically makes electrical measurements of thoracic and abdominal movement. For example, sensor 26 may comprise two or more skin electrodes, which are driven by console 28 to make a plethysmographic measurement of the change in impedance or inductance between the electrodes as a result of the patient's respiratory effort. (It is also possible to use the ECG electrodes for this purpose.) Alternatively, the respiration sensor may comprise a belt, which is placed around the patient's chest or abdomen and senses changes in the body perimeter. Additionally or alternatively, measurement of flow through the patient's airway may be used for respiration sensing. For example, the air flow from the patient's nose and/or mouth may be measured using a pressure cannula, thermistor, or CO2 sensor. Any other suitable respiration sensor known in the art may also be used, in addition to or instead of the above sensor types.

Additionally or alternatively, console 28 may gather signals from an existing set of sensors coupled to patient 22. For example, while patient 22 is undergoing Holter monitoring, as is known in the art, the monitored physiological signals may also be used for detection and analysis of stress events, as described hereinbelow. As another example, implantable cardiac devices, such as pacemakers and ICDs, typically sense the patient's ECG and are capable of transmitting telemetry signals out to a suitable receiver. Such implantable devices sometimes include motion sensors, as well, such as an accelerometer, whose output may also be used, along with the ECG, in stress analysis. Additionally or alternatively, the implantable device may generate and transmit impedance-based respiration measurements (known in the art as “minute ventilation”).

Console 28 may process and analyze the ECG, respiration and other signals locally, using the methods described hereinbelow. In the present embodiment, however, console 28 is coupled to communicate over a network 30, such as a telephone network or the Internet, with a diagnostic processor 32. This configuration permits sleep studies to be performed simultaneously in multiple different locations. Processor 32 typically comprises a general-purpose computer with suitable software for carrying out the functions described herein. This software may be downloaded to processor 32 in electronic form, or it may alternatively be provided on tangible media, such as optical, magnetic or non-volatile electronic memory. Processor 32 analyzes the signals conveyed by console 28 in order to identify sleep stages of patient 22 and to display the results of the analysis to an operator 34, such as a physician.

The following table summarizes exemplary parameters that may be monitored and analyzed in system 20 for purposes of testing the response of patient 22 to spontaneous stress events that occur during sleep. Typically, only a subset of these parameters is monitored, depending on available sensing modalities and clinical indications. The parameters in the table are listed by way of example, and other parameters may also be monitored, using any suitable monitoring modalities known in the art.

TABLE I
MONITORED PARAMETERS

| 1. | Parameters derived from respiration including respiration calculated | |
|---|---|---|
| | from ECG, photoplethysmograph, minute ventilation (implantable) | |
| | or auscultation: | |
| | a. | Envelope |
| | b. | Respiration rate |
| 2. | ECG and heart rate: | |
| | a. | RR interval, QQ interval, heart rate or any other |
| | | indicator of heart rate coming from ECG, |
| | | photoplethysmograph or auscultation |
| | b. | Ischemia indicator such as ST level (ST depression) |
| | c. | Any abnormal beat morphology (especially PVC) |
| | d. | Envelope of the above |
| 3. | Photoplethysmograph: | |
| | a. | Derived heart rate |
| | b. | Derived respiration rate |
| | c. | Envelope |
| | d. | Saturation and/or its envelope |
| 4. | Pulse transit time (PTT): | |
| | a. | Time elapsed between ECG feature (such as R location) |
| | | and a subsequent photoplethysmograph feature (such |
| | | as maximal derivative) |
| | b. | Time elapsed between signal features from two |
| | | photoplethysmograph sensors, such as between ear and |
| | | finger |
| 5. | Motion: | |
| | a. | Leg movement |
| | b. | Actigraph |

| | |
|---|---|
| 6. | Arousals derived from EEG, motion, or noise in any other signal |

In some embodiments, system 20 is used in conjunction with therapeutic devices, such as a respirator 36. For example, detection and evaluation of periodic breathing patterns, as described hereinbelow, may be used to control the pressure and/or gas mixture administered by the respirator. Further details of treatment modalities of this sort are described in a U.S. provisional patent application entitled, “Respiratory Support of Co-Morbid Apnea Patients,” filed Jan. 30, 2006, which is assigned to the assignee of the present patent application and whose disclosure is incorporated herein by reference.

FIG. 1B is a schematic, pictorial illustration showing an alternative configuration of system 20 using a novel sensing device 40, in accordance with an embodiment of the present invention. Device 40 comprises a belt, which fits around the patient's body (typically around the chest), with a sensor unit 42 connected to console 28. The sensor unit measures respiratory motion of the chest (in terms of strain in the belt, for example) and also comprises plethysmographic sensor 44, which is held against the body by the belt and measures oxyhemoglobin saturation percutaneously. Sensor 44 typically comprises an optical radiation source and a detector, which measures the reflected radiation as a function of wavelength, as is known in the art. Device 40 can be worn comfortably by the patient during sleep at home, in the clinic or in the hospital, and provides sufficient data to permit diagnosis of apnea type (central or obstructive) and assessment of the patient's stress response, in the manner described hereinbelow.

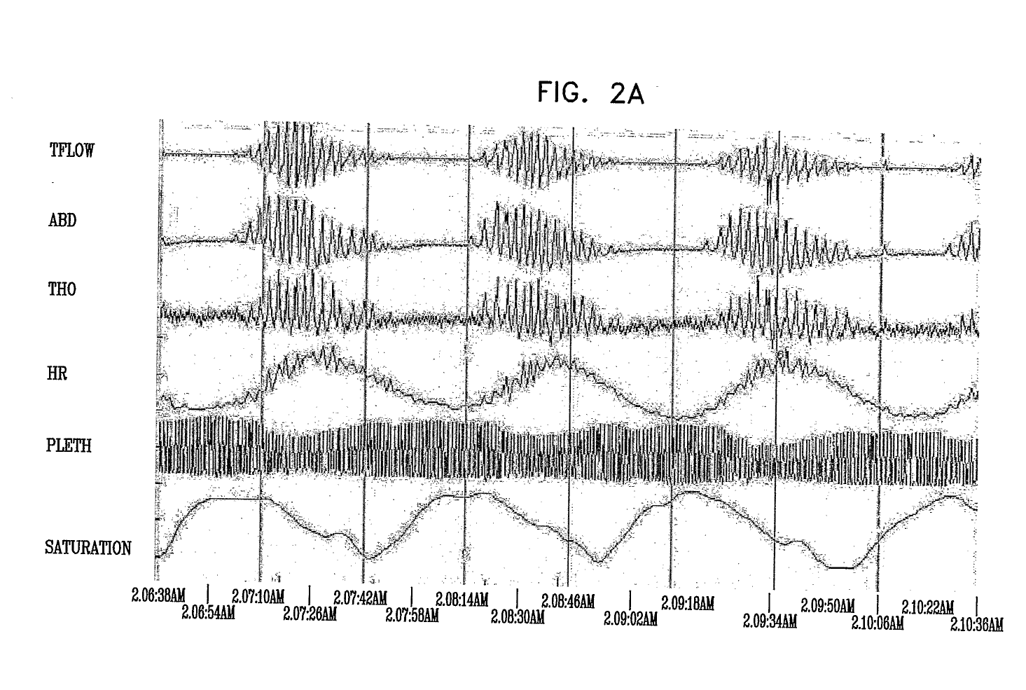

FIGS. 2A and 2B are plots of signals collected from patients during sleep using the monitoring devices of system 20, in accordance with an embodiment of the present invention. The upper trace in both figures represents air flow measured through the patient's airway (TFLOW, using a thermistor to sense the flow; other means known in the art, such as a pressure sensor or capnograph, may alternatively be used for this purpose). The next two traces show abdominal (ABD) and thoracic (THO) movement, respectively. These top three traces show well-defined periodic breathing patterns: periods of hypoventilation, in which there is little or no thoracic and abdominal movement, alternating with periods of intense hyperventilation. The near absence of thoracic and abdominal movements during the intervals of hypoventilation in FIG. 2A indicates that this patient was suffering from CSA, while the patient of FIG. 2B suffered from OSA.

The three lower traces in FIGS. 2A and 2B show heart rate (HR), plethysmograph (PLETH), and oxygen saturation (SAT) measurements, respectively. These signals likewise exhibit periodic behavior that tracks the periodic respiration activity shown by the top three traces. It will be observed that the periodic waveforms in FIG. 2A appear to be roughly symmetrical, rising gradually to a peak amplitude over a certain rise time and then dropping off in like fashion. (The term “symmetry” in this context refers to mirror-image symmetry of the signal about a vertical line taken through a local maximum or minimum in the signal.) The periodic waveforms in FIG. 2B, on the other hand, are asymmetrical, rising sharply and then falling off more gradually.

These observations with respect to the symmetry of the periodic breathing patterns apply both to the slowly-varying heart rate and saturation signals and to the envelopes of the other, rapidly-varying signals shown by the other traces. The term “envelope” in this context typically means a signal derived from the local minima and/or local maxima of another signal, with or without smoothing (by convolution or resampling, for example). “Envelopes” may also be derived by other mathematical operations known in the art, such as application of Hilbert transforms. The inventor has found that periodic breathing patterns associated with CSA generally tend to be more symmetrical than the patterns associated with OSA, presumably due to the different physiological mechanisms that are involved in the different types of apneas.

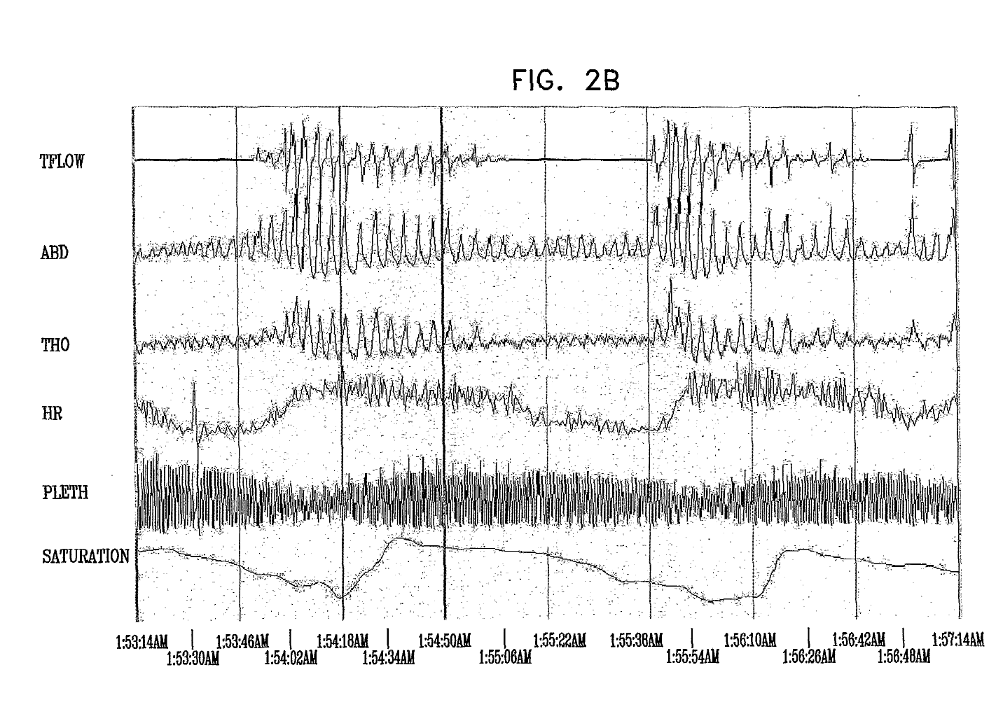

FIG. 3 is a flow chart that schematically illustrates a method for detection and analysis of stress events, in accordance with an embodiment of the present invention. Processor 32 analyzes the physiological parameters gathered from patient 22 during sleep in order to identify spontaneous stress events, at an event identification step 50. Stress events can generally be identified as transient, non-stationary segments in the signal recordings. One type of stress event, as described above, is a respiratory event, such as an apnea or hypopnea, which may take the form of a periodic breathing episode. Other stress events detected by processor 32 may include patient movements and cardiac events, such as transient atrial fibrillation, tachycardia, premature ventricular contractions (PVC), bigemini or trigemini.

Various methods may be used to detect stress events automatically. One possible method is to divide the monitored signal into quasi-stationary segments. Methods of adaptive segmentation that may be applied to physiological signals for this purpose (particularly in the context of sleep analysis) are described in detail in the above-mentioned US 2004/0230105 A1. Briefly, the adaptive segmentation process divides the time series into segments, each of which is characterized by quasi-stationary behavior. “Quasi-stationary” means that certain statistical properties of each segment, such as spectral amplitude variations, are contained within predefined bounds. Those segments of the time series that are not quasi-stationary over at least a predefined minimum duration may be identified as transient events, which may include stress events.

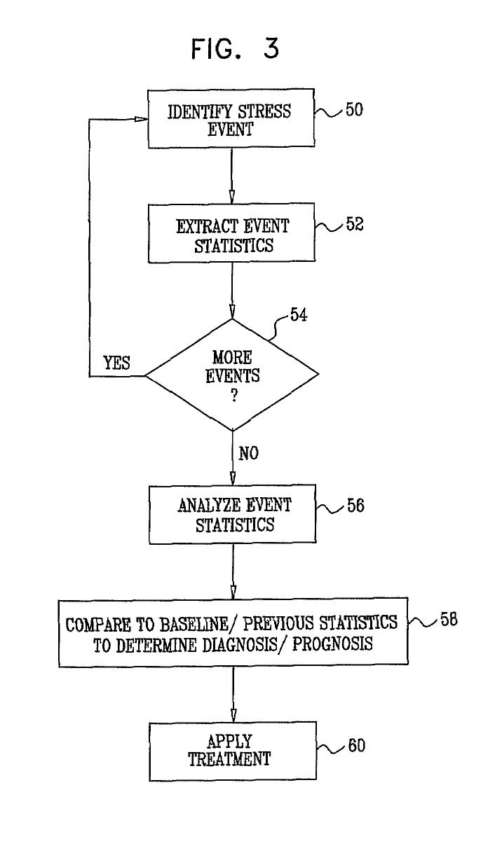

Upon identifying a stress event, processor 32 processes the signals collected during the period of the event and for a predefined period thereafter in order to gather event statistics, at a statistics extractions step 52. The statistics gathered at this step may include, for example, the duration of the event, the variance and frequency of changes in physiological parameters during and after the event, and the time required for recovery of these parameters to steady-state values after the event. An exemplary method for extraction of relevant parameters from periodic breathing events is described below with reference to FIG. 4. Processor 32 continues to process the signals recorded by system 20 until a predetermined time period has been covered and no more events are found, at a monitoring completion step 54.

After collecting all relevant events from a given patient monitoring session, processor 32 analyzes the event statistics, at an analysis step 56. Some typical statistical characteristics are listed by way of example in Table II:

TABLE II
STRESS CHARACTERISTICS

Effort level:
Duration
Type (such as central vs. obstructive apnea)
Extrema (such as maximum HR)
Trends (gradient)
Cardiac/respiratory synchronization loss
Recovery:
Time/trend to return to normal of each signal
Onset, slope
Cardiac-respiratory-saturation synchronization time
Change after predefined time interval

Other statistical characteristics, as are known in the art, may be used in addition to or instead of those listed above. Processor 32 may compare the statistics to those of other patients with known disease histories, as well as to baseline statistics generated for this specific patient during previous monitoring sessions, at a comparison step 58. This statistical comparison may be used in diagnosing the patient's current medical condition, as well as in assessing and updating the patient's prognosis.

The results of the preceding analysis may then be used in determining the course of treatment to be applied to the patient, at a treatment step 60. For example, a heart failure patient found to be suffering from predominantly obstructive apnea might be treated by administration of continuous positive airway pressure (CPAP) respiration, while drug therapy or controlled administration of a gas mixture of O₂ and CO₂ might be used to treat predominantly central apnea. Improvement over time in the periodic breathing patterns of the patient suffering from central apnea could be an indication that the drug therapy has succeeded in palliating the patient's condition, whereas degradation over time could lead to administering some form of respiration support.

Additionally or alternatively, when processor 32 detects a potentially dangerous situation, such as a prolonged apnea or other deterioration relative to the patient's baseline statistics, the processor may alert (and awaken) the patient and/or alert the patient's medical caregiver.

FIG. 4 is a plot showing parameters used in a method applied by processor 32 for analyzing periodic breathing patterns, in accordance with an embodiment of the present invention. The method may be applied, for example, to the heart rate and/or saturation signals shown in FIGS. 2A and 2B, as well as to the envelopes of the other respiration-related signals. It models the successive hyperventilation and hypoventilation segments in the respiration-related signals as a sequence of parabolic forms, opening upward and downward in alternation. Each parabola has an apex (x₀,y₀) and may be tilted at an angle θ. The parabola may thus be expressed in terms of transformed coordinates as follows:

$$\{ \begin{matrix}( \begin{matrix}u \\ v\end{matrix} ) = ( \begin{matrix}cos θ & sin θ & {x}_{0} \\ - sin θ & cos θ & {y}_{0}\end{matrix} ) ( \begin{matrix}x \\ y \\ - 1\end{matrix} ) \\ v = {( σ u )}^{2}\end{matrix} \qquad ( 1 )$$

Rearranging the terms in equation (1) gives:

(x cos θ+y sin θ)²σ²−2(x cos θ+y sin θ)(σ²x₀)−(σ²x₀+y₀)=−x sin θ+y cos θ (2)

Defining a new set of parameters ā=(a₁, a₂, a₃ such that:

$$\overline{a} = ( \begin{matrix}{a}_{1} \\ {a}_{2} \\ {a}_{3}\end{matrix} ) = ( \begin{matrix}{σ}^{2} \\ {σ}^{2} {x}_{0} \\ {σ}^{2} {x}_{0} - {y}_{0}\end{matrix} ) \qquad ( 3 )$$

equation (2) can be written as a system of over-determined linear equations for a given set of sample points {xᵢ,yᵢ}:

[(x cos θ+y sin θ)²a₁−2(x cos θ+y sin θ)a₂−a₃=x sin θ+y cos θ (4)

Now denoting:

$$P = ( \begin{matrix}{( {x}_{1} cos θ + {y}_{1} sin θ )}^{2} & - 2 ( {x}_{1} cos θ + {y}_{1} sin θ ) & - 1 \\ & ⋮ & \\ {( {x}_{n} cos θ + {y}_{n} sin θ )}^{2} & - 2 ( {x}_{n} cos θ + {y}_{n} sin θ ) & - 1\end{matrix} ) \\ \overline{b} = ( \begin{matrix}- {x}_{1} sin θ + {y}_{1} cos θ \\ ⋮ \\ - {x}_{n} sin θ + {y}_{n} cos θ\end{matrix} ) \qquad ( 5 )$$

We can rewrite equation (4) as:

Pā= b̅ (6)

or (using equation (3)):

$$( \begin{matrix}{σ}^{2} \\ {σ}^{2} {x}_{0} \\ {σ}^{2} {x}_{0} - {y}_{0}\end{matrix} ) = {( {P}^{T} P )}^{- 1} ( {P}^{T} \overline{b} ) \qquad ( 7 )$$

If the result of the fit gives |σ|<ε (for a predetermined small value ε) or |σ|>τ (for a large value τ), the segment in question is approximated by a line, rather a parabola. In other words, the processor fits the sample points to a line [yᵢaxᵢ+b , and θ=arctan a.

The method described above may be used to find the best fit for any arbitrary value of θ. The optimal value of θ is typically chosen as the one that gives the minimal squared error. (Alternatively, other error measurements may be used.) The optimal θ may be found using nonlinear optimization techniques that are known in the art, such as the Newton-Raphson method.

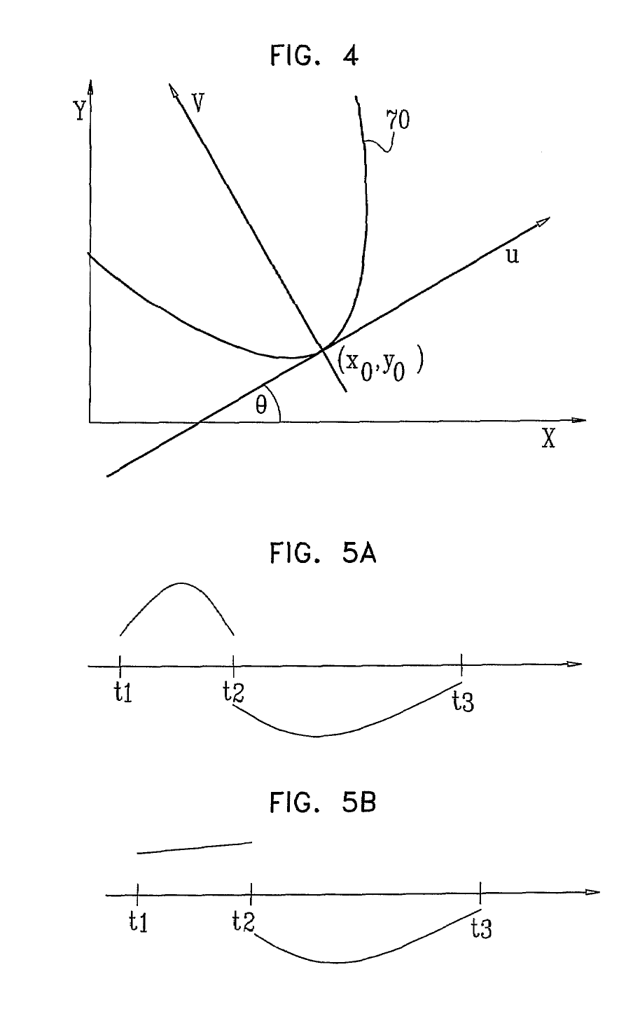

Reference is now made to FIGS. 5A and 5B, which are plots that schematically illustrate curves that have been fitted to periodic respiration signals, in accordance with an embodiment of the present invention. FIG. 5A corresponds to a single cycle of a pattern of CSA (as shown in FIG. 2A, for example), while FIG. 5B corresponds to a single cycle of a pattern of OSA (FIG. 2B). The hyperventilation portion of each cycle is between times t₁ and t₂, while the hypoventilation portion is between times t₂ and t₃. The curves are parabolic, as explained above, with the exception of the linear hyperventilation portion in FIG. 5B.

To complete the parabolic fit of equation (1), a length function is defined as:

L(t₁,t₃)=(t₃−t₁−T)² (8)

wherein T is the typical apnea cycle. In Cheyne-Stokes breathing, a typical value is T=60 sec. A hyperventilation/hypoventilation length ratio function is also defined as follows:

$$R ( {t}_{1} , {t}_{2} , {t}_{3} ) = {( \frac{{t}_{2} - {t}_{1}}{{t}_{3} - {t}_{1}} - δ )}^{2} \qquad ( 9 )$$

wherein typically

$$δ = \frac{1}{2} .$$

For each time interval [tᵢ,tᵢ₊₁], i.e. xᵢε[tᵢ,tᵢ₊₁], i=1, . . . , n, the fitted parabola in the interval can be expressed as:

$$\{ \begin{matrix}( \begin{matrix}{u}_{i} \\ {v}_{i}\end{matrix} ) = ( \begin{matrix}cos θ & sin θ & {x}_{0} \\ - sin θ & cos θ & {y}_{0}\end{matrix} ) ( \begin{matrix}{x}_{i} \\ {y}_{i} \\ - 1\end{matrix} ) \\ {\tilde{v}}_{i} = {( σ {u}_{i} )}^{2}\end{matrix} \qquad ( 10 )$$

The least-mean-square fitting error is then given by:

$${ɛ}^{2} ( {t}_{i} , {t}_{i + 1} ) = \frac{1}{n} {\sum }_{\{ j \vert {x}_{j} ∈ [ {t}_{i} , {t}_{i + 1} ] \}} {( {\tilde{v}}_{j} - {v}_{j} )}^{2} \qquad ( 11 )$$

or in the case of a line, rather than a parabola:

$${ɛ}^{2} ( {t}_{i} , {t}_{i + 1} ) = \frac{1}{n} {\sum }_{\{ j \vert {x}_{j} ∈ [ {t}_{i} , {t}_{i + 1} ] \}} {( {y}_{j} - ( {ax}_{j} + b ) )}^{2} \qquad ( 12 )$$

Processor 32 thus finds the fit that minimizes:

aL(t₁,t₃)+βR(t₁,t₂,t₃)+λ₁ε²(t₁,t₂)+λ₂ε²(t₂,t₃) (13)

Typically, in this formula, the following parameter values are used:

$$α = \frac{1}{{T}^{2}} ; β = \frac{1}{{δ}^{2}} ; {λ}_{i} = {( {max}_{{x}_{i} ∈ [ {t}_{i} , {t}_{i + 1} ]} {y}_{i} - {min}_{{x}_{i} ∈ [ {t}_{i} , {t}_{i + 1} ]} {y}_{i} )}^{- 2}$$

Alternatively, other fitting parameter values may be used.

Further alternatively, other methods of calculation known in the art may be used to extract shape characteristics from respiration-related signals and to assess the symmetry of periodic breathing waveforms. For example, the waveforms may be convolved with symmetric and anti-symmetric kernels (such as Gaussian and first derivative of a Gaussian), and the extrema of the results may be then be located. As another example, not only parabolas, but also other analytical functions, such as splines or other polynomials, may be fitted to the waveforms.

After fitting has been completed, processor 32 may extract features of diagnostic importance from the resulting parabolas and lines (or other shapes if used). These parameters include the time values (t₁, t₂, t₃), as well as the fitting types (parabola or line) and values of θ and σ. The waveform is considered to be symmetrical—and thus indicative that the apnea or hypopnea is of predominantly central origin—if θ is less than a predefined threshold value. Large values of θ are indicative that the apnea or hypopnea is of predominantly obstructive origin, while intermediate values may indicate mixed influence of central control and obstruction

Other features of importance may include the maximum and minimum values of the monitored parameters during each interval (hyperventilation and hypoventilation) in the periodic breathing episode, and the relative times at which these values occur. These features are indicative of the response of the patient's body to the stress caused by hypoventilation and hyperventilation during the periodic breathing episode. Exemplary features, as well as their relative values during the hyperventilation and hypoventilation intervals, are listed in the table below:

TABLE III
FEATURES OF PERIODIC BREATHING EPISODES

Respiration rate: hyperpnea ≧ hypopnea
Heart rate: hyperpnea ≧ hypopnea
RR and other intervals: hyperpnea ≦ hypopnea
Photoplethysmograph envelope: hyperpnea ≦ hypopnea
PTT: hyperpnea ≦ hypopnea
Motion, arousal: hyperpnea ≧ hypopneas

The time difference between each feature in one signal and the corresponding feature in another signal may also be measured, along with the coherence between signals or their representations. Clustering may be used to group intervals having similar features for purposes of diagnosis and assessment of prognoses. Methods of clustering that may be used for this purpose are described, for example, by Jain et al., in “Data Clustering: A Review,” ACM Computing Surveys 31:3, pages 264-323, which is incorporated herein by reference.

Central sleep apnea and Cheyne-Stokes breathing are thought to be associated with increased sensitivity to CO₂, as explained, for example, by Javaheri, in “A Mechanism of Central Sleep Apnea in Patients with Heart Failure,” New England Journal of Medicine, 341:13 (1999), pages 949-954, which is incorporated herein by reference. Therefore, quantifying central sleep apnea (including Cheyne-Stokes respiration of central etiology), as described above, can also provide a biomarker that is associated with the rate of increase of ventilation per unit of increase in CO₂ production (VE/VCO₂ slope), which is commonly evaluated in cardiopulmonary stress test. Respiratory gas exchange variables of this sort have clinical and prognostic importance in diagnosing and treating CHF patients, as described, for example, by Corra et al., in “Ventilatory Response to Exercise Improves Risk Stratification in Patients with Chronic Heart Failure and Intermediate Functional Capacity,” American Heart Journal 143:3 (March, 2002), pages 418-426, which is incorporated herein by reference. A different sort of biomarker can be obtained from the patient's response to stress that is induced by obstructive apnea.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A computer-implemented method for monitoring a patient, comprising:
receiving a signal associated with respiration of the patient during sleep;
processing the signal to detect a pattern of periodic breathing;
extracting from the signal a shape characteristic of the pattern; and
classifying an etiology of the periodic breathing responsively to the shape characteristic.

2. The method according to claim 1, wherein classifying the etiology comprises determining the periodic breathing to be predominantly central or obstructive in origin.

3. The method according to claim 2, wherein extracting the shape characteristic comprises computing a parameter that is characteristic of a symmetry of the pattern.

4. The method according to claim 3, wherein determining the periodic breathing comprises classifying the periodic breathing as predominantly central in origin if the pattern is symmetrical.

5. The method according to claim 1, wherein receiving the signal comprises receiving an indication of least one of a flow through an airway of the patient, a movement of an abdomen or thorax of the patient, a heart rate of the patient, a respiration rate of the patient, a blood flow of the patient, a blood oxygen saturation level of the patient, and a pulse transit time (PTT) of the patient.

6. The method according to claim 1, wherein receiving the signal comprises receiving a photoplethysmographic signal from an organ of the patient.

7. The method according to claim 6, wherein receiving the signal comprises fastening a belt holding a reflective photoplethysmographic sensor around the patient, and measuring both a respiratory movement and a blood oxygen saturation level of the patient using the belt and photoplethysmographic sensor.

8. The method according to claim 1, wherein the pattern comprises a hyperventilation interval and a hypoventilation interval, and wherein extracting the shape characteristic comprises fitting first and second waveforms to the hyperventilation and hypoventilation intervals, respectively.

9. The method according to claim 8, wherein the first and second waveforms comprise analytical functions.

10. The method according to claim 9, wherein the analytical functions comprise polynomial waveforms.

11. The method according to claim 9, wherein the analytical functions comprise tilted parabolic waveforms.

12. The method according to claim 8, and comprising extracting features from the signal responsively to the first and second waveforms, and selecting a treatment to administer to the patient based on the extracted features.

13. A computer-implemented method for monitoring a patient, comprising:
receiving physiological signals from the patient during sleep;
processing at least one of the signals to detect a spontaneous stress event; and
analyzing one or more of the signals following the stress event so as to evaluate a stress response of the patient.

14. The method according to claim 13, wherein receiving the physiological signals comprises receiving an indication of least one of a flow through an airway of the patient, a movement of an abdomen or thorax of the patient, a heart rate of the patient, a respiration rate of the patient, a blood flow of the patient, a blood oxygen saturation level of the patient, and a pulse transit time (PTT) of the patient.

15. The method according to claim 13, wherein the spontaneous stress event comprises at least one of an apnea and a hypopnea.

16. The method according to claim 15, wherein analyzing the one or more of the signals comprises assessing a periodic breathing pattern associated with the at least one of the apnea and the hypopnea.

17. The method according to claim 13, wherein receiving the physiological signals comprises receiving a photoplethysmographic signal from an organ of the patient.

18. The method according to claim 17, wherein receiving the physiological signals comprises fastening a belt holding a reflective photoplethysmographic sensor around the patient, and measuring a movement of the thorax and at least one of a blood flow of the patient and a blood oxygen saturation level of the patient using the belt and photoplethysmographic sensor.

19. The method according to claim 18, wherein receiving the physiological signals comprises receiving an electrocardiogram (ECG) signal from a Holter monitor coupled to the patient.

20. The method according to claim 13, wherein analyzing the one or more of the signals comprises detecting a change in the signals relative to a baseline, and alerting at least one of the patient and a medical caregiver of the change.

21. The method according to claim 13, wherein receiving the physiological signals comprises collecting the signals from the patient at a first location where the patient is sleeping, and transmitting the signals over a communication network, and
wherein analyzing the one or more of the signals comprises receiving the one or more of the signals over the communication network at a second location, remote from the first location, and analyzing the received signals at the second location.

22. Apparatus for monitoring a patient, comprising:
a sensor, which is coupled to produce a signal associated with respiration of the patient during sleep; and
a diagnostic processor, which is arranged to process the signal to detect a pattern of periodic breathing, to extract from the signal a shape characteristic of the pattern, and to classify an etiology of the periodic breathing responsively to the shape characteristic.

23. The apparatus according to claim 22, wherein the processor is arranged to determine the periodic breathing to be predominantly central or obstructive in origin.

24. The apparatus according to claim 23, wherein the shape characteristic comprises a parameter that is characteristic of a symmetry of the pattern.

25. The apparatus according to claim 24, wherein the processor is arranged to classify the periodic breathing as predominantly central in origin if the pattern is symmetrical.

26. The apparatus according to claim 22, wherein the signal is indicative of least one of a flow through an airway of the patient, a movement of an abdomen or thorax of the patient, a heart rate of the patient, a respiration rate of the patient, a blood flow of the patient, a blood oxygen saturation level of the patient, and a pulse transit time (PTT) of the patient.

27 The apparatus according to claim 22, wherein the sensor comprises a photoplethysmographic sensor.

28. The apparatus according to claim 27, and comprising a belt, which is configured to hold the photoplethysmographic sensor and to be fastened around the patient, wherein the photoplethysmographic sensor comprises a reflective photoplethysmographic sensor, which is arranged to measure at least one of a blood flow and a blood oxygen saturation level of the patient, and the sensor is also coupled to the belt so as to measure a respiratory movement of the patient using the belt.

29. The apparatus according to claim 28, and comprising a Holter monitor, which is coupled to convey an electrocardiogram (ECG) signal from the patient to the diagnostic processor.

30. The apparatus according to claim 22, wherein the pattern comprises a hyperventilation interval and a hypoventilation interval, and wherein the processor is arranged to fit first and second waveforms to the hyperventilation and hypoventilation intervals, respectively.

31. The apparatus according to claim 30, wherein the first and second waveforms comprise analytical functions.

32. The apparatus according to claim 31, wherein the analytical functions comprise polynomial waveforms.

33. The apparatus according to claim 31, wherein the analytical functions comprise tilted parabolic waveforms.

34. The apparatus according to claim 30, wherein the processor is arranged to extract features from the signal responsively to the first and second waveforms, and to determine a treatment to administer to the patient based on the extracted features.

35. Apparatus for monitoring a patient, comprising:
a sensor, which is coupled to receive physiological signals from the patient during sleep; and
a diagnostic processor, which is arranged to process at least one of the signals to detect a spontaneous stress event, and to analyze one or more of the signals following the stress event so as to evaluate a stress response of the patient.

36. The apparatus according to claim 35, wherein the physiological signals comprises an indication of least one of a flow through an airway of the patient, a movement of an abdomen or thorax of the patient, a heart rate of the patient, a respiration rate of the patient, a blood flow of the patient, a blood oxygen saturation level of the patient, and a pulse transit time (PTT) of the patient.

37. The apparatus according to claim 35, wherein the spontaneous stress event comprises at least one of an apnea and a hypopnea.

38. The apparatus according to claim 37, wherein the processor is arranged to assess a periodic breathing pattern associated with the at least one of the apnea and the hypopnea.

39. The apparatus according to claim 35, wherein the sensor comprises a photoplethysmographic sensor.

40. The apparatus according to claim 39, and comprising a belt, which is configured to hold the photoplethysmographic sensor and to be fastened around the patient, wherein the photoplethysmographic sensor comprises a reflective photoplethysmographic sensor, which is arranged to measure at least one of a blood flow and a blood oxygen saturation level of the patient, and the sensor is also coupled to the belt so as to measure a respiratory movement of the patient using the belt.

41. The apparatus according to claim 40, wherein the processor is arranged to detect a change in the signals relative to a baseline, and to alert at least one of the patient and a medical caregiver of the change.

42. The apparatus according to claim 40, wherein the sensor is operative to collect the signals from the patient at a first location where the patient is sleeping, and comprising a console, which is coupled to receive the signals from the sensor and to transmit the signals over a communication network, and
wherein the processor is coupled to receive the signals over the communication network at a second location, remote from the first location, and to analyze the received signals at the second location.

43. Apparatus for monitoring a patient, comprising:
a belt, which is adapted to be fastened around a body of the patient; and
a sensor unit, which is coupled to the belt so as to sense a respiratory motion of the patient, and which comprises a photoplethysmographic sensor, which is held against the body by the belt and is arranged to measure at least one of a blood flow and a blood oxygen saturation level of the patient.

44. The apparatus according to claim 43, wherein the belt is configured to be fastened around a thorax of the patient, and wherein the photoplethysmographic sensor comprises a reflective sensor.
